(19) United States
(12) Patent Application Publication
(10) Pub. No.: US 2015/0182605 A1
(43) Pub. Date: Jul. 2, 2015

(54) Ultra-Low Dose Lysostaphin for Treating MRSA

(71) Applicant: StaphRx, LLC, Bethesda, MD (US)

(72) Inventor: Gerald W. Fischer, Bethesda, MD (US)

(73) Assignee: StaphRx, LLC, Bethesda, MD (US)

(21) Appl. No.: 14/584,101

(22) Filed: Dec. 29, 2014

Related U.S. Application Data
(60) Provisional application No. 61/921,443, filed on Dec. 28, 2013.

(51) Int. Cl. A61K 38/48; C12N 9/52; A61K 45/06

(52) U.S. Cl. CPC A61K 38/4886; A61K 45/06; C12N 9/52; C12Y 304/24075; A61K 2039/505

(57) ABSTRACT

The present invention is directed to compositions and methods for treating diseases and disorders of patients and, in particular, compositions and methods for treating Staphylococcus infections of patients with ultra-low doses and altered forms of lysostaphin, and synergistic combinations of lysostaphin plus additional conventional treatments such as antibiotic and/or antibody treatment. The invention is also directed to detecting and identifying altered forms of lysostaphin that possess increased efficacy against infections as compared to wild-type lysostaphin, and forms that generate a minimal or no immune response in a patient. The invention is also directed to method of manufacturing these altered forms of lysostaphin.

### REFERENCE TO RELATED APPLICATIONS

This application claims priority to U.S. Provisional Application No. 61/921,443 of the same title and filed Dec. 28, 2013, the entirety of which is hereby incorporated by reference.

### BACKGROUND

1. Field of the Invention

The present invention is directed to compositions and methods for treating diseases and disorders of patients and, in particular, compositions, uses of the compositions and methods for treating Staphylococcus infections of patients with ultra-low doses and altered forms of lysostaphin, or with lysostaphin and synergistic combinations of lysostaphin and conventional treatments such as antibiotics, antibodies or other enzymes. The invention is also directed to detecting and identifying altered forms of lysostaphin that possess increased efficacy against infections as compared to wild-type lysostaphin, and forms that generate a minimal or no immune response in a patient. The invention is also directed to method of manufacturing these altered forms of lysostaphin.

2. Description of the Background

Staphylococcus aureus (SA) is a major cause of severe infections of animals and people. In humans, a skin infection may rapidly progress from a mild local infection or wound to sepsis, multi-organ system failure, shock and death within hours. Surgical patients, diabetics and premature infants are also highly susceptible to infection and individuals with catheters, artificial valves and other foreign bodies may develop colonization of these devices that are very difficult treat. Over the last several years, SA has become resistant to many key antibiotics including the first line penicillins (e.g. multi-drug resistant Staphylococcus aureus or generally MRSA). These organisms have spread around the world and MRSA is a major cause of both community and hospital acquired infections. There is a great need to develop new approaches to treating MRSA to save lives and reduce the cost of this severe and difficult to treat disease process.

Severe SA infections, such as endocarditis, can be difficult to treat even with standard antibiotics such as oxacillin or vancomycin. Drug resistant SA infections (MRSA/Vancomycin-resistant Staphylococcus aureus or generally VRSA) are even more challenging to eradicate. Staphylococcal infections are a major concern in all clinical settings, particularly procedures that involve implantable objects. Infections can become tenacious and, due to the nature of Staphylococci, the bacteria tend to form as layers or biofilms in and around the implanted objects. Once a Staphylococcal biofilm has formed within the body, disruption of the film becomes problematic and antibiotic resistance is common.

Due to emerging antibiotic resistance and a greater use of implantable objects, an immediate and largely unmet need exists for an effective treatment against SA infections.

### SUMMARY OF THE INVENTION

The present invention overcomes the problems and disadvantages associated with current strategies and designs and provide new tools and methods for treating Staphylococcus infections.

One embodiment of the invention is directed to compositions for administration to a patient in need thereof comprising a therapeutically effective amount of lysostaphin. Preferably the lysostaphin is administered at from 5 μg to 0.5 mg per kg of patient body weight and the patient in need thereof has or is at risk of acquiring a Staphylococcus infection. Preferably the effective amount of lysostaphin comprises one or more doses that provide a serum (or plasma) or tissue level of lysostaphin at from 0.001 μg/ml to 50 μg/ml, and more preferably from 0.01 μg/ml to 20 μg/ml. Preferably the lysostaphin is wild-type lysostaphin isolated from Staphylococcus staphylolyticus or from Staphylococcus staphylolyticus cultured from selective media or from another organism or cell type (e.g., E. coli, insect cells, and mammalian cells transfected with lysostaphin sequences). Preferably the selective media comprises glucose and also preferably the lysostaphin is recombinant lysostaphin isolated from E. coli bacteria or another cell type. Preferably the lysostaphin has one or more amino acids or one or more amino acid modifications that differ from wild-type lysostaphin and the lysostaphin has a greater efficacy against Staphylococcus infection as compared to wild-type lysostaphin and/or wherein the lysostaphin has a reduced or no immune response when administered to the patients in need thereof as compared to wild-type lysostaphin.

Another embodiment of the invention is directed to lysostaphin therapy for prophylaxis or treatment of active or suspected infections. Preferably lysostaphin therapy further comprises a secondary therapy for the patient in need thereof that is synergistic with the lysostaphin. Synergistic therapies include use of agents such as, for example, other enzymes, cell wall active agents, chemicals, peptides, and/or antibodies. Preferred agents include one or more of the chemical forms and derivatives of penicillin, antibiotics such as, for example, nicin, bacteriocins, amoxicillin, augmentin, polymyxins, isoniazid, rifampin, ethambutol, Pyrazinamide, aminoglycosides, colistins, cycloserine, autolysin, bacitracin, cephalosporin, vancomycin, and/or beta lactam. Preferably the antibiotics are administered in one or more doses to the patient in need thereof at an effective dose that is lower than the recommended dose for administration of the antibiotic alone and the composition further comprises a pharmaceutically acceptable carrier such as, for example, one or more of oil, fatty acids, lipids, polymers, carbohydrates, gelatin, solvents, saccharides, buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents or an immunological inert substance, a carrier designated as generally recognized as safe (GRAS), or a combination thereof.

Another embodiment of the invention is directed to methods for treating a Staphylococcus comprising administering to a patient in need thereof a composition comprising lysostaphin preferably wherein the lysostaphin is administered at from 5 μg to 0.5 mg per kg of patient body weight. Preferably the composition is administered orally, aerosolized (e.g. preferably as nanoparticles to the lungs), encapsulated, prepared as slow-release, intravenously or subcutaneously or injected into a site of infection, the composition is coated onto an object to be inserted into the body of the patient wherein the object is inserted into an area of the body that is infected and/or sequestered from the patient's immune system. Preferably the composition has reduced negative effects or increased positive effects for the patient as compared with conventional therapy. Preferably the reduced negative effects include one or more of reduced toxicity and reduced immunogenicity and the enhanced positive effects include one or more of increased efficacy and enhanced clearance from a patient system.

Other embodiments and advantages of the invention are set forth in part in the description, which follows, and in part, may be obvious from this description, or may be learned from the practice of the invention.

### DESCRIPTION OF THE DRAWING

FIG. 1 Antibody values of mouse sera binding to lysostaphin-coated plates.

FIG. 2 Antibody values of mouse sera binding to lysostaphin-coated plates.

FIG. 3 Histogram of change in % T of S. aureus at 650 nm following treatment with S. staphylolyticus lysostaphin.

FIG. 4 Histogram of change in % T (at 650 nm) of a neat of S. aureus type 5 solution following treatment with recombinant or natural lysostaphin.

FIG. 5 Histogram of change in % T (at 650 nm) of a 1:2 dilution of S. aureus type 5 solution following treatment with recombinant or natural lysostaphin.

FIG. 6 Growth of SA5 (ATCC 49521) treated with Nafcillin (25 mg/ml) +/−Lysostaphin (0.0123 units/ml).

FIG. 7 Growth of SA5 (ATCC 49521) treated with Nafcillin (12.5 mg/ml) +/−Lysostaphin (0.0123 units/ml).

FIG. 8 Growth of SA5 (ATCC 49521) +/−Lysostaphin (0.0123 units/ml) without Nafcillin.

FIG. 9 Growth of SA5 (ATCC 49521) treated with Oxacillin (25 mg/ml) +/−Lysostaphin (0.0123 units/ml).

FIG. 10 Growth of SA5 (ATCC 49521) treated with Oxacillin (12.5 mg/ml) +/−Lysostaphin (0.0123 units/ml).

FIG. 11 Growth of SA5 (ATCC 49521) +/−Lysostaphin (0.0123 units/ml) without

Oxacillin.

FIG. 12 Effect of Lysostaphin (0.0123 units/ml) on growth of SA5 49521 in presence and absence of Nafcillin.

FIG. 13 Effect of Lysostaphin (0.0123 units/ml) on growth of SA5 49521 in presence and absence of Oxacillin.

### DESCRIPTION OF THE INVENTION

Lysostaphin is a well-know bacteriocin secreted by cells of staphylococcal bacteria, typically and preferably isolated from S. simulans. The production of lysostaphin is well known and the enzyme is commercially available (U.S. Pat. No. 3,278,378). The lysostaphin gene encodes a preproenzyme of Mr 42,000. The NH₂-terminal sequence of the preproenzyme is composed of a signal peptide followed by seven tandem repeats of a 13-amino acid sequence. Conversion of pro-lysostaphin to the mature enzyme occurs extracellularly in cultures of S. simulans and involves removal of the NH₂-terminal portion of the proenzyme that contains the tandem repeats. The high degree of homology of the repeated regions would suggest that the repeats arose from duplication of a 39-base-pair sequence of DNA. Recsei et al, Proc Natl Acad Sci USA. Mar; 84(5):1127-31 (1987).

Lysostaphin is a 27 kD glycylglycine endopeptidase that functions by cleaving the pentaglycine bridge of bacterial cell walls. As such, lysostaphin is a potent anti-Staphylococcal enzyme. Lysostaphin therapy for experimental endocarditis and sepsis utilized large IV doses; standard anti-SA antibiotics can be added as well (U.S. Pat. No. 8,198,231). For treating serious SA infections (such as endocarditis with bacteremia) in animal models, typical doses of lysostaphin used were from 10-50 mg/kg and for up to 6 weeks. Treatment was focused on direct lysis of SA cells by lysostaphin, and thus, similar regimens have been extrapolated for treating severe SA infections in humans. These high intravenous doses may have deleterious effects related to infusing a protein/enzyme into the blood stream such as inducing antibodies, kidney disease and/or vasculitis.

It has been surprisingly discovered that, to treat SA infections and minimize side effects due the lysostaphin itself, ultra-low dose lysostaphin therapy can utilize a variety of dosing schedules and synergistic strategies. These include, but are not limited to sequential pulses of very small amounts of lysostaphin to disrupt cell walls and promote immune clearance, while controlling the blood lysostaphin level. Preferably antibiotics are given at selected intervals as well to synergize with the local or systemic levels of lysostaphin. Ultra-low dose lysostaphin therapy can be administered in a manner and provides a therapeutically effective treatment for infections caused by MSSA, MRSA, coagulase-negative staphylococci (CNS) such as sepsis and endocarditis. Ultra-low dose therapy comprises therapeutic administration at below conventional doses and evokes fewer or no side effects and allows for a lower cost of treatment. The benefits of reduced dosing include, but are not limited to a reduced impact to the immune system, kidneys, livers, heart, lungs and other major organs and systems of a body. With ultra-low dose therapy, blood pressure is less affected (which may otherwise increase or decrease), clearance of medication from the patient's system is accelerated, immune system response (as in allergic response and/or inflammation) may be reduced or eliminated, as well as all known side effects of any medication such as, for example, anemia, hemophilia and other bleeding disorders, platelet deficiency and other clotting disorders, risks of blood-derived and other cancers, and all risks and side effects of the particular therapy. The compositions and methods of the invention are useful to treat microbial infection that is caused by an organism that is generally susceptible to an antibiotic irrespective of the acquisition or development of resistance to the antibiotic.

It was also surprisingly discovered that synergy of other active agents with lysostaphin could be better achieved with low doses of lysostaphin. Although higher doses of lysostaphin would result in greater overall percent killing of microorganisms, with lower doses a synergy could be observed with a secondary active agent. Preferred secondary active agents include but are not limited to agents that are active against cell wall construction, cell wall lysis, replication, transcription, translation, polymerases and other specific enzymes, and other major functions associated with bacteriostatic and/or bactericidal activity.

One embodiment of the invention is directed to compositions containing ultra-low doses of lysostaphin to prevent or to treat infection such as, preferably, infections by Staphylococcus and other organisms. Preferably these doses contain additional antibiotics or antibacterial compounds (bacteriostatic or bactericidal), or possibly antibodies or other compounds to generate or enhance the patient's immune response to an infection. Also preferably, the compositions contain a pharmaceutically acceptable carrier that is a recognized and approved by an appropriate authority (e.g., U.S. Food and Drug Administration, European Medicine's Agency).

Composition of the invention may also contain or be administered with a secondary therapy, such as, for example, bacteriostatic or bactericidal therapy, antibody therapy (e.g., anti-Staphylococcus antibodies such as monoclonal or polyclonal antibodies or antibody fragments), and/or antibiotic therapy (e.g. one or more of vancomycin, teicoplanin, telavancin, clindamycin, lincomycin, linezolid, rifampin, polymyxin B and C, neomycin, cefalexin, ceftaroline fosamil, ceftobiprole), treatment with another medication, or a combination of these secondary treatments. The combination of lysostaphin with a secondary therapy creates a synergy that improves treatment outcomes or allows for a reduction in amount, dosage and/or concentration as compared to conventional dosing of the secondary therapy alone. Preferred classes of antibiotics that can be combined with lysostaphin therapy and preferably ultra-low dose lysostaphin therapy for treatment of patients are listed in Table 1.

TABLE 1
Useful Classes of Antibiotics

| | | |
|---|---|---|
| Aminoglycosides | Lincosamides | Polypeptides |
| Ansamycins | Lipopeptide | Quinolones |
| Bacteriosins | Lantbiotics | Rifampin |
| Carbacephem | Macrolides | Sulfonamides |
| Carbapenems | Nitrofurans | Tetracyclines |
| Cephalosporins | Oxazolidonones | Anti-Mycobacterial compounds |
| Glycopeptides | Penicillins | |

Medications listed in Table 1 are preferably administered at a reduced concentration, as compared to conventional and individually recommended dosages, when administered in combination with lysostaphin therapy. The combinations produce surprising synergistic effects on the host which is preferably a human patient or non-human patient (e.g., preferably mouse or other mammal), often clearing an infection with little or no ill effects to the patient as can be seen with higher doses of lysostaphin and/or higher doses of the one or more antibiotics alone.

Another embodiment of the invention is directed to methods for administering to a patient in need thereof, one or more doses of a composition containing ultra-low doses of lysostaphin and/or preferred forms of lysostaphin that are altered as compared to wild type or conventional lysostaphin (recombinant amino acid sequence of lysostaphin has additional sequences as compared to the natural amino acid sequence). Preferably, the antimicrobial activity of the composition (e.g. the lysostaphin and/or the lysostaphin plus the synergistic compounds of the composition) has an activity of 50% or greater, more preferably 70% or greater, more preferably 80% or greater, more preferably 90% or greater, more preferably 95% or greater, and more preferably 99% or greater. Preferably the ratio of active to inactive forms of lysostaphin is two or greater, three or greater, five or greater, seven or greater, or ten or greater. Maximization of activity allows for the administration of minimal dosages and thereby reduces the risk of immunogenicity.

Preferably these one or more doses are administered simultaneously or over a course of time (before or after administration of lysostaphin) with the secondary therapy such as, for example, antibiotics, antibacterial chemicals and chemical compounds, and other enzymes (e.g. chemical forms and derivatives of penicillin, amoxicillin, augmentin, bacteriosin, polymyxin, colisti, cycloserine, autolysin, bacitracin, cephalosporin, rifampin, vancomycin, beta lactam), or possibly antibodies or other compounds to generate or enhance an immune response to the infection. Preferably the antibiotic functions synergistically with the lysostaphin of the invention to provide an efficient and effective preventative or treatment of an infection. Also preferably, antibiotics are not needed in bacteriostatic or bactericidal quantity, which is not only advantageous with regard to expense, availability and disposal, these lower dosages do not necessarily encourage development of resistance to the same degree, together a tremendous and surprising benefit of the invention. Lysostaphin compositions may be administered to treat infections that have sensitivity or resistance to certain antibiotics. Treating infections that are sensitive to antibiotics prevents the development and/or the rise of antibiotic resistant forms. Also preferably, administration of antibiotics with lysostaphin may be alternated and/or staggered to prevent and/or reduce the risk of developing resistance to the antibiotic or to the lysostaphin molecule.

Doses may also be as international units. One international unit (IU) is about 3.1 μg and will reduce the turbidity (A620) of a suspension of S. aureus cells from 0.250 to 0.125 in 10 minutes at pH 7.5 at 37° C. in a 6.0 ml reaction mixture.

The compositions of the invention including but not limited to ultra-low doses of lysostaphin and lysostaphin plus secondary therapies, may be administered orally, parenterally (e.g., intravenously), topically, transdermally, by intramuscular injection or injection to the site of infection, or by intraperitoneal injection, or the like, although oral administration is generally preferred. Administration can be continuous (e.g., drip, infusion, delayed or gradual release), or site specific, such as to sites of a patient that are sequestered from the patient's immune system (e.g., areas of the central nervous system {CNS}, areas behind the blood-brain barrier, peripheral nerve canals, optical cavities and optical nerves), and to areas of that body the receive only reduced or limited immunological activity (e.g., areas with limited blood flow). Administration may also be by aerosol formulations, encapsulation, liposomal formulations and may include the coating of objects that are placed fully or partially into the patient, such as the coating of catheters, drainage tubes (e.g., for CNS drainage). Methods to coat medical objects and compositions formulated to maintain coatings to objects are well-known to those skilled in the art (Boston Scientific Corporation of Natick, Mass. and see U.S. Pat. Nos. 8,034,119 and 8,597,673).

The composition may also comprise lysostaphin coupled with an antibody that directs or drives the antibody to a specific site, such as for example, the site of an active infection within the patient. Preferably the antibody and the lysostaphin are covalently coupled and also preferably, the antibody and the lysostaphin may be directed to a site that contains enzymes that cleave the coupling releasing the lysostaphin intact or functionally active. The antibody may have activity against the infection (e.g., an anti-Staph antibody) or have another beneficial effect to the patient, or the antibody is used solely to direct or target the placement and/or activity of the lysostaphin, such as, for example, to a specific organ, tissue, cell surface, or site within the body of the patient.

The amount of lysostaphin in compositions of the invention administered to patients is dependent on, for example, the weight of the patient and the mode of administration. When administered to specific sites, such as for example, by injection to a site of infection, injection to a confined region or the patient's body, or aerosolization to the lungs, dosages can be substantially reduced from a total weight-based amount or total plasma concentration. Generally, the dosage will be in the range of preferably between about 1 μg to 1.0 mg per kg of patient body weight, more preferably between about 2 μg to 0.5 mg per kg of patient body weight, more preferably between about 5 μg to 0.1 mg per kg of patient body weight, and more preferably between about 25 μg to 0.05 mg per kg of patient body weight. Dosages not based on patient total weight preferably provide an effective serum (or plasma) or tissue level of lysostaphin at from about 0.001 μg/ml to about 1.0 mg/ml, also preferably from about 0.005 μg/ml to about 500 μ/ml, also preferably from about 0.01 μg/ml to about 100 μg/ml, also preferably from about 0.02 μg/ml to about 50 μg/ml, also preferably from about 1.0 μg/ml to about 10 μg/ml and also preferable combinations therein. Dosages may be administered as a single bolus, every 8 hours, every 12 hours (bid), daily (qd), every other day (qod), or the frequency of administration empirically determined by one skilled in the art as appropriate to treat the infection. Administration is typically sufficient for 5 days, for 7 days, for 10 days, for 14 days, or can be for shorter or longer periods of time as determined by one skilled in the art.

Also preferably the lysostaphin compositions of the invention are aerosolized to a degree that is effective, for example, when treating infections of the lungs. Preferably compositions are aerosolized to nanoparticles or particles of about 1-3 microns that are able to reach deep into lung tissue. Such small particles are also taken up by macrophages and delivered to the site of the infection. The amount of lysostaphin administered is dependent on, for example, the weight of the patient, the severity of the infection, the state of the patient's immune system, and/or the mode of administration.

Also preferably, the lysostaphin compositions of the invention may be encapsulated such as, for example, as liposomes, or as nanoparticles, or prepared as emulsions or microspheres, conjugated with organic or non-organic compounds that may or may not include immune stimulating agents, complexed with compounds that couple multiple lysostaphin molecules together, or with biodegradable coating for slow-release and/or timed-release formulations. Biodegradable coatings are preferably polymers or co-polymers such as, for example, carbohydrates, lipids, fatty acids, peptides, proteins, nucleic acids and combinations thereof.

Depending on the intended mode of administration, the pharmaceutical compositions may be in the form of solid, semi-solid or liquid dosage forms, such as, for example, tablets, suppositories, pills, capsules, powders, liquids, suspensions, sprays or the like, preferably in unit dosage form suitable for single administration of a precise dosage. The lysostaphin may be encapsulated for immediate or slow release (e.g. carbohydrate or sugar coatings), aerosolized to the site of the infection as, for example, nanoparticles. The compositions may include, as noted above, an effective amount of lysostaphin in combination with a pharmaceutically acceptable carrier and, in addition, may include other medicinal agents, pharmaceutical agents, carriers, adjuvants, diluents, and the like. For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, etc., an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known or apparent to those skilled in this art (e.g., see Remington's Pharmaceutical Sciences. 15th Edition. Edited under the direction of Arthur Osol and John E. Hoover. Mack Publishing Co., which is hereby incorporated by reference). For oral administration, fine powders or granules may contain diluting, dispersing, and/or surface active agents, and may be presented in water or in a syrup, in capsules or sachets in the dry state, or in a non-aqueous solution or suspension wherein suspending agents may be included, in tablets wherein binders and lubricants may be included, or in a suspension in water or a syrup. Where desirable or necessary, flavoring, preserving, suspending, thickening, or emulsifying agents may be included. Tablets and granules are preferred oral administration forms, and these may be coated.

Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system, such that a constant level of dosage is maintained (e.g., U.S. Pat. No. 3,710,795, which is hereby incorporated by reference).

Another embodiment of the invention is directed to methods for the production of altered forms of lysostaphin that show greater efficacy against the infection. Production methods preferably include isolation of lysostaphin from cultures of bacterial containing a selective grown medium. Preferred growth media ingredients include, but are not limited to serum, carbohydrate (e.g., glucose, sucrose, fructose), one or more bacterial cell growth factors, one or more vitamins and/or one or more essential or non-essential amino acids. Growth of bacterial strains is preferable at optimum proliferation temperatures (typically 37° C.) and more preferably at temperatures reduced from optimal. Also preferred is growth with selective pressure to promote a desired phenotypic characteristic. By way of a non-limiting example, bio variant Staphylococcus simulans (subsp. Staphylolyticus) is preferably grown at about 37° C., more preferably about 32° C. or less, more preferably about 30° C. or less, and more preferably about 28° C. or less. Increased cultivations temperatures as well as variation in temperature can also generate expression of altered forms of lysostaphin.

The form (e.g., 3D structure, immunogenicity, nucleic acid and/or amino acid sequence, post-translation modification) and activity of lysostaphin can be altered by the methods of production. For example, lysostaphin activity, function and/or immunogenicity can be increased or decreased as compared to wild-type (wt.) forms by adjusting, for example, the pH, the temperature, or the composition of the growth media (e.g., glucose level, amino acid choice and composition, serum type and/or percentage, etc.) of the culture. Isolation and/or purification of lysostaphin from cultures grown with selected ingredients and/or at reduced temperatures generates variants that have one or more altered amino acid sequences, one or more altered chemical modifications, and/or altered folding or 3-D configurations. These altered forms of lysostaphin show a reduced capacity to generate an immune response in patients upon injection and/or provide increased efficacy of the lysostaphin against infection. Alterations of the sequence can be created with recombinant techniques as well. Alterations of chemical modifications can be directed by the particular cultures in which the enzyme is cultivated such as, for example, cell cultures of E. coli, Staphylococcus simulans (staphylolyticus), Lactococcus lactis, Lactobacillis fermentum, or Lactobacillus rhamnosus. Altered form of lysostaphin are isolated and purified from these cultures as the same manner as wild-type or natural lysostaphin.

Another embodiment of the invention is directed to compositions comprising altered forms of lysostaphin. The preferred amino acid sequence is of wild-type or normal lysostaphin and is derived from Staphylococcus simulans (SubSp.staphylolyticus). Altered forms of lysostaphin of the invention may preferably contain one or more amino acid substitutions, amino acid modifications, one or more amino acid deletions and/or one or more amino acid additions to the sequence. Altered forms possess an altered 3-D structure, configuration and/or folding to the protein molecule thereby providing certain advantages. For example, altered forms of lysostaphin preferable have a greater efficacy and an increased potency against infection and/or faster acting function. Also preferable, these altered forms are minimally or completely non-reactive to the patient's immune system (decreased or no immunogenicity) causing the generation of minimal or no humoral, cellular or inflammatory response. As a direct result, treatment of patients with an altered form of lysostaphin of the invention would produce reduced side effects as compared to previously known lysostaphin treatment, toxicity may be reduced and clearance enhanced. The burden to a patient's organ system such as the liver or kidneys, would be reduced which may allow for increased dosages and even greater efficacy. Altered lysostaphin therapy can also be combined with other therapies such as, for example, antibody and/or antibiotic therapy as described herein.

The following examples illustrate embodiments of the invention, but should not be viewed as limiting the scope of the invention.

### EXAMPLES

### Example 1

Ultra-low dose lysostaphin (ULDL) (0.005-0.5 mg/kg) is given intravenously or at the site of MRSA infection in combination with one or more selected antimicrobials. These antimicrobials include, but are not limited to nafcillin, oxacillin, methicillin, vancomycin, gentamicin, quinolones, erythromycin, rifampin, polymixins and antimicrobial peptides. A tissue or foreign body infection treated with a short burst of 1-6 ultra-low doses over 1-3 days in combination with one or more antimicrobials eradicates bacteremia and improves survival. In addition, a continuous ULDL infusion over 12-28 hours clears bacteremia and infection.

### Example 2

ULDL enhances immunity to MRSA when phagocytic cells come in contact with MRSA in the presence of ULDL alone (0.005-0.5 μg/ml) and in combination with one or more selected antimicrobials at or below their mic/mbc. The phagocytes have increased phagocytosis and or greater MRSA killing.

### Example 3

Two lysostaphin products are injected into mice (one produced in S. simulans, subspecies S. staphylolyticus {natural}, and one produced in E. coli designated S. simulans {recombinant}). As lysostaphin is an immunogenic protein, when injected (into people or animals) antibodies are generated that may cause side effects after previous exposure. Anti-lysostaphin antibodies are produced in the serum by both products. Despite the fact that both products induced similar anti-lysostaphin antibodies, the antibodies induced by both products had markedly higher binding to recombinant lysostaphin compared to non-recombinant lysostaphin. One animal in the non-recombinant lyso-group produced almost no antibody response. Surprisingly, these data demonstrate that the method of production can provide a lysostaphin molecule that has increased binding to anti-lysostaphin antibodies. The recombinant lysostaphin is more reactogenic and more potent than conventionally produced lysostaphin. Thus, this data shows that production methods can specifically alter immunogenic properties and enhance potency to provide effective therapy at ultra-low doses and minimize side effects.

### Example 4

Assay of mouse sera on plates coated with lysostaphin from simulans expressed as recombinant in E coli and coated with natural lysostaphin from S. staphylolyticus. Mice received a primary immunization (subcutaneous) with 10 μg recombinant lysostaphin from E. coli (Sigma-Aldrich) (animal numbers 8603, 8604) or 10 μg of natural lysostaphin from S. staphylolyticus (Sigma-Aldrich) (animal numbers 8621-8623). Primary immunization was administered and boosted about three weeks later. No adjuvant was used for the immunizations.

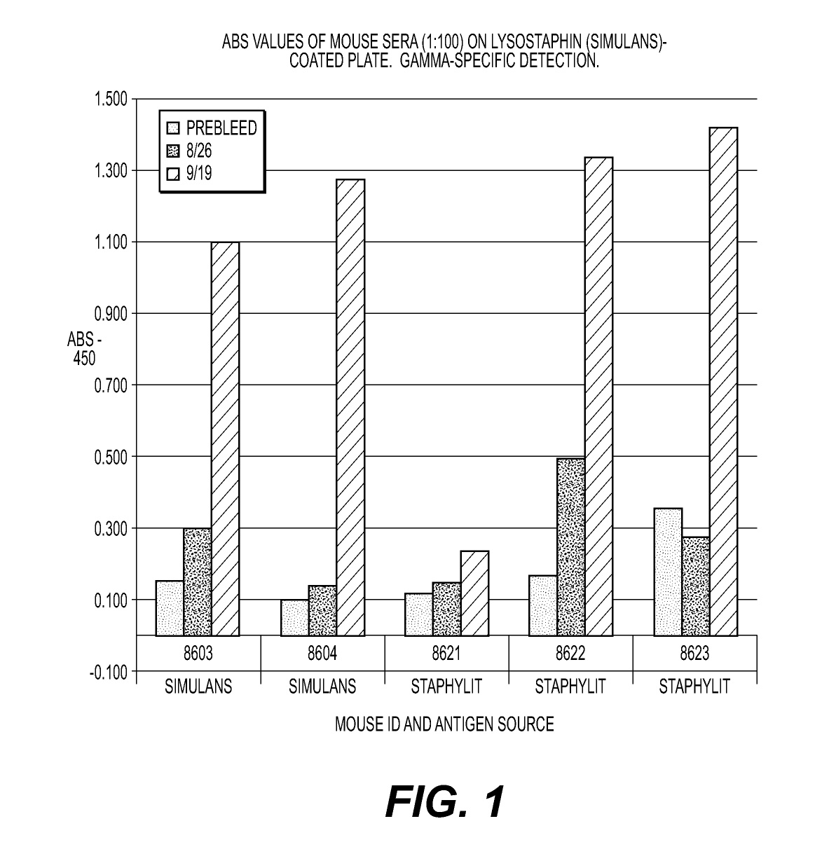

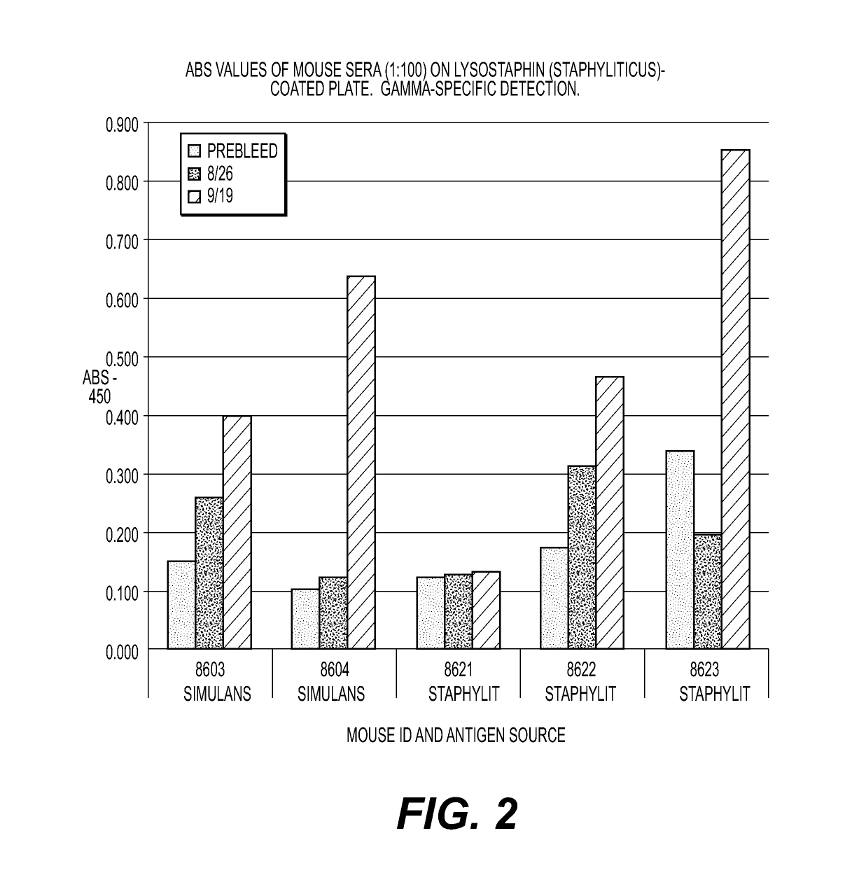

Serum samples were tested using gamma-specific detection (FIGS. 1 and 2). Bars represent binding seen with normal serum (first bar), serum after the first injection (second bar), and serum after the boost injection three weeks later (third and in most cases largest bar). The results on wells coated with simulans lysostaphin (FIG. 1) were stronger than the results on wells coated with staphylolyticus lysostaphin (FIG. 2), regardless of the immunogen. Mouse 8621 was poorly reactive on both coating antigens. Note that each graph is scaled to 1.500 on the y-axis for comparison purposes. Based on these results another boost is needed to raise the titers to a level optimal for fusion (i.e. absorbance greater than 1.000 at a serum dilution of 1:10000 or higher).

### Example 5

50 μl of S. aureus type 5 (ATCC 49521) was plated onto blood agar plates and incubated overnight. The resulting bacterial lawn was transferred to 30 mls of TSB (tryptic soy broth; Remel cat 112740) and incubated at 37° C. for about 60 minutes at 225 rpm.

The percent transmission (% T) of the bacterial suspension following incubation was about 7%. The % T was adjusted to 53% by addition of 100 μl of bacterial suspension to 4500 μl of TSB (in five individual tubes) and the % T was measured in each tube. An additional tube contained TSB alone.

Tube #1 received no lysostaphin.

Tube #2 was designated to receive 550 μl of recombinant lysostaphin, but the lysostaphin was erroneously added to Tube #3, and thus, Tube #2 was discarded.

Tube #3 received 550 μl recombinant lysostaphin (from a 2,000 μg/ml stock solution) for a final concentration of about 220 μg/ml (this tube erroneously received lysostaphin dose intended for tube #2 (i.e. 500 μl of lysostaphin stock) plus the 50 μl of lysostaphin originally intended for this tube.

Tube #4 received 500 μl of natural lysostaphin from S. staphylolyticus (from a 2,000 μg/ml stock solution) for a final concentration of about 200 μg/ml.

Tube #5 received 50 μl of natural lysostaphin from S. staphylolyticus (from a 2,000 μg/ml stock solution) for a final concentration of about 20 μg/ml.

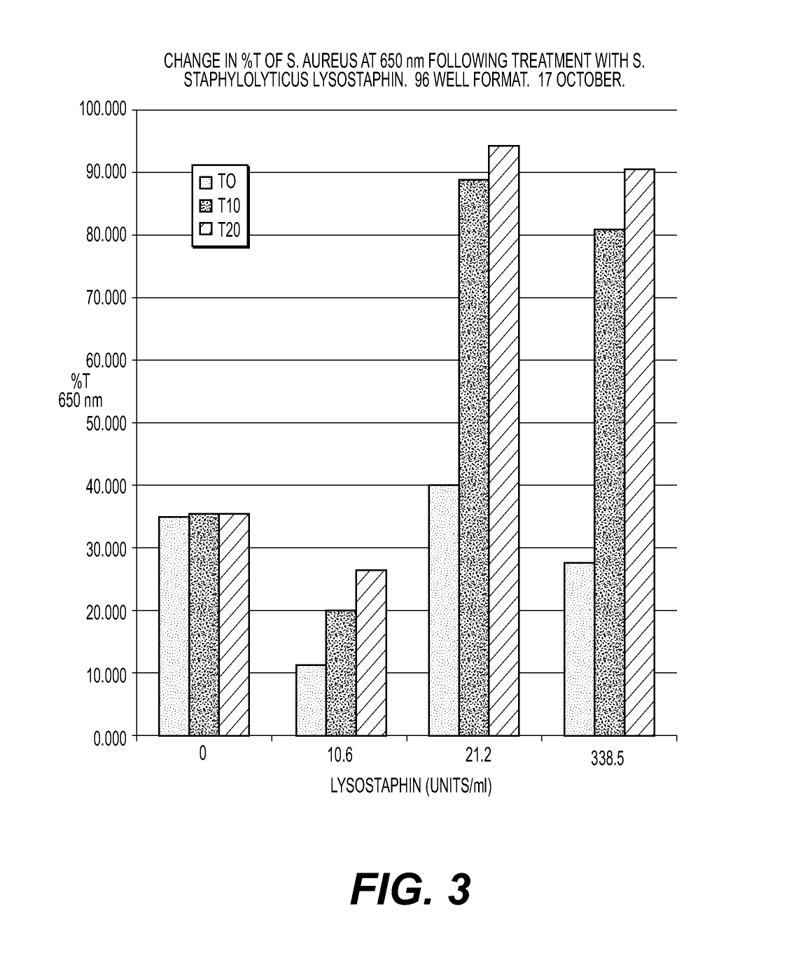

The tubes were vortexed, incubated at 37° C. for 10 minutes at 150 rpm and the % T for each determined (see FIG. 3 which is summarized in Table 2).

TABLE 2

| Tube | Treatment | Starting % T | Ending % T |
|---|---|---|---|
| Untreated | None/no bacteria | 100 | 100 |
| 1 | Bacteria alone | 53.0 | 50.6 |
| 3 | recombinant 220 μg/ml | 54.2 | 94.0 |
| 4 | natural 200 μg/ml | 51.8 | 94.6 |
| 5 | natural 20 μg/ml | 52.4 | 95.0 |

An increase in the % T indicates killing of bacteria. Both lysostaphins were highly likely active against this SA5.

A second assay was performed in which the SA5 and lysostaphin were tested in a 96-well configuration. For this assay, 100 μl of the SA5 solution was added to wells followed by 100 μl of the lysostaphin solution. % T was obtained at TO and the plate incubated at 37° C. for 10 minutes with gentle rotation. The % T was obtained and the results compared.

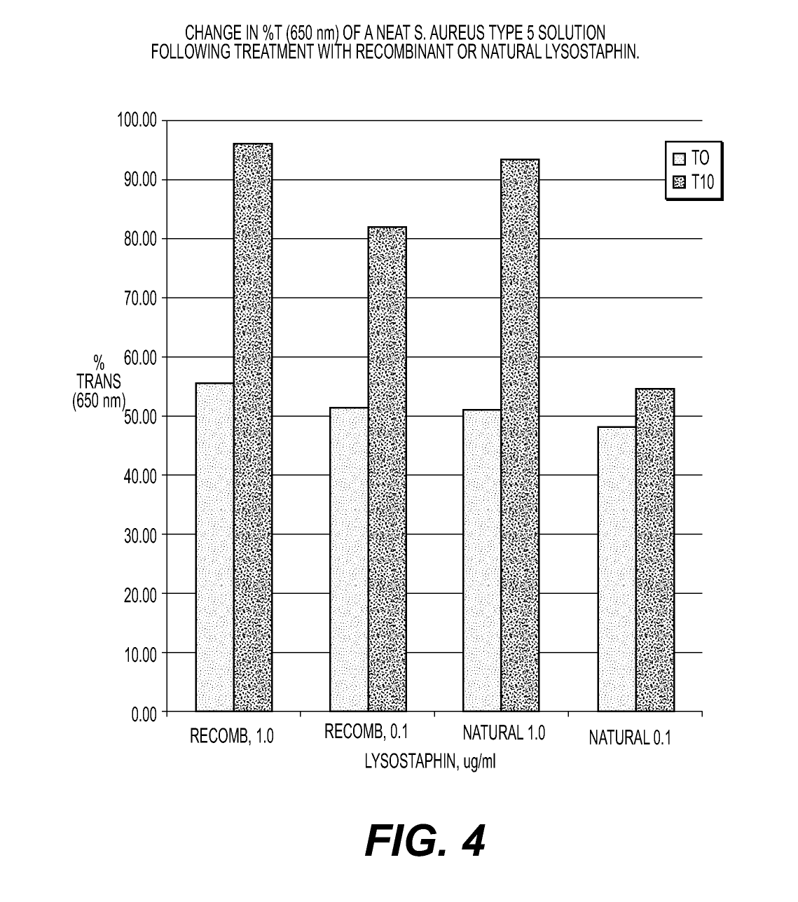

As shown in FIG. 4, treatment of the neat solution of SA5 with either lysostaphin at 1 μg/ml resulted in a dramatic increase in the % T. When lysostaphin was used at 0.1 μg/ml, the killing obtained with the recombinant lysostaphin remained strong evidenced by the increase in the % T. However, with natural lysostaphin the % T increased only slightly, suggesting that the recombinant lysostaphin is more active than the natural product on a weight to weight basis. Review of the COA's for both products shows that the recombinant lysostaphin has an enzymatic activity of greater than 3,000 units per mg. Thus, the differences obtained at 0.1 μg/ml are not surprising since the recombinant material is approximately 6-7 fold more active.

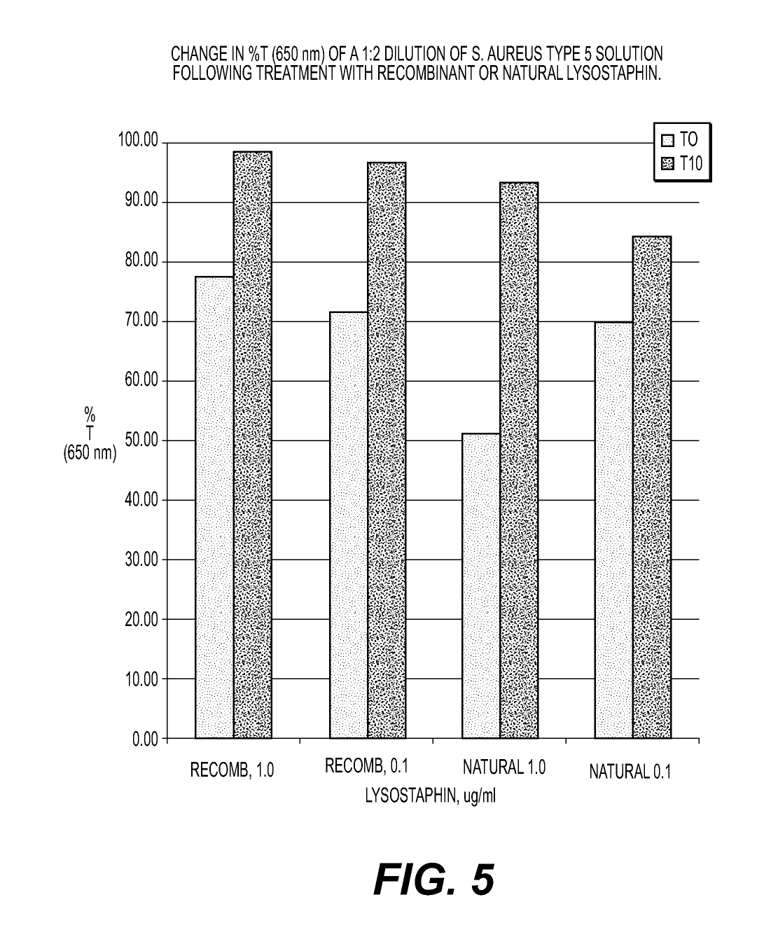

When the assay was run with SA5 at a dilution of 1:2 the results were similar (FIG. 5) to those obtained with neat bacteria, although changes in % T for the natural lysostaphin at 0.1 μg/ml shows a greater change than obtained when neat SA5 were used.

### Example 6 Effect of Antibiotics and Lysostaphin on Growth of SA5 (ATCC 49521)

It was determined whether low levels of lysostaphin would act synergistically with Nafcillin or Oxacillin to kill an SA5 (ATCC 49521). The study was performed in a 96-well dish to allow titration of lysostaphin and antibiotics. The end point was % T. Plating of bacteria from the assay plate was not done.

Fifty μl of SA5 (ATCC 49521) was reconstituted from a frozen stock and grown overnight on a blood agar plate at 37° C. Bacteria were transferred from the blood agar plate (approximately 25% of the lawn) into 30 mls of TSB and grown for 2 hours at 37° C. on a rotating shaker set at 250 rpm. The initial % T of the bacterial suspension was 24.2%. After incubation the % T was 11.5%. A 1:4 dilution of this suspension in TSB gave a % T of ˜50% in a 96 well dish.

For the assay, lysostaphin (Sigma Cat L7386; lot 063M4011V; 3387.5 units/ml stock in water) was diluted in three-fold dilutions (in TSB) from 3 units/ml (row A) to 0.0123 units/ml (row G) in 50 μl/well. The wells in row H received TSB alone. This plate was designated the assay plate. In a separate 96-well plate (i.e. the dilution plate), Nafcillin (Sigma Cat N3269; lot SLBF8230V, 50 mg/ml in water) was diluted in TSB to 25 mg/ml and a second dilution of 12.5 mg/ml was also prepared (columns 1 and 2 of the dilution plate). Oxacillin (Sigma Cat 01002; lot SLBC9948V; 50 mg/ml in water) was diluted to 25 mg/ml in TSB and a second dilution of 12.5 mg/ml was also prepared (columns 4 and 5 of the dilution plate).

Fifty μl of the antibiotic solutions were transferred to the corresponding wells of the assay plate containing the lysostaphin solution. 50 μl of TSB alone was transferred to columns 3 and 6 (i.e. no antibiotic controls) of the assay plate. The combination of antibiotics and lysostaphin yielded a volume of 100 μl. 125 μl of S. aureus 49521, diluted 1:2 in TSB was then added to all wells of the plate, except wells H1, H2, H4 and H5, which served as the blank well for determining the % T. After addition of the bacteria, and between each determination of the % T, the plate was incubated at 37° C. with gentle rotation (˜50 rpm). The % T was then determined at 2 and 15 minutes after addition of the bacteria, and again at 5 hours and 22 hours after addition of the bacteria.

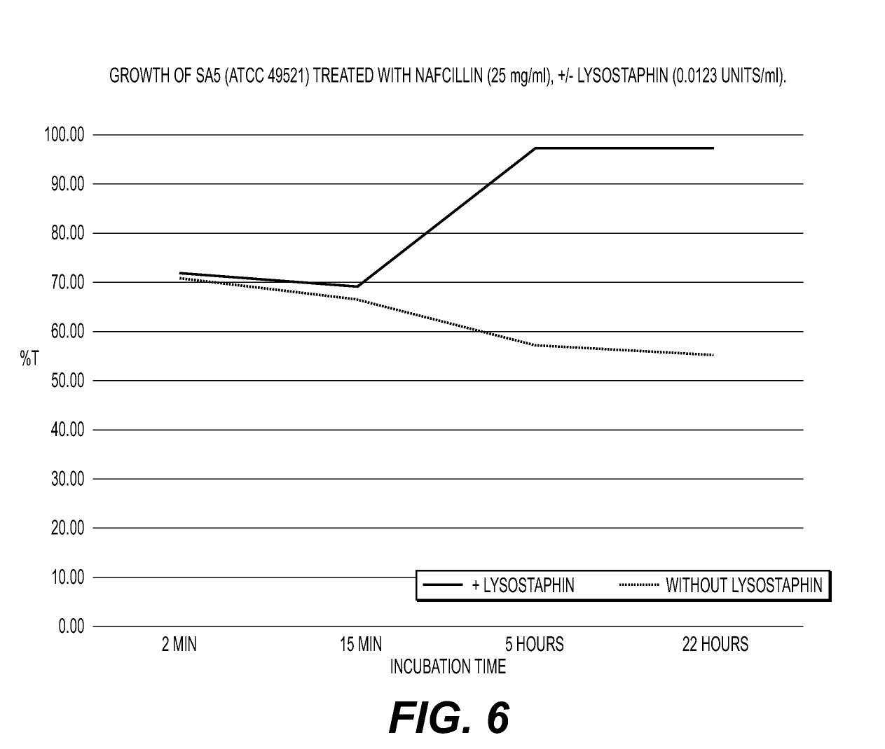

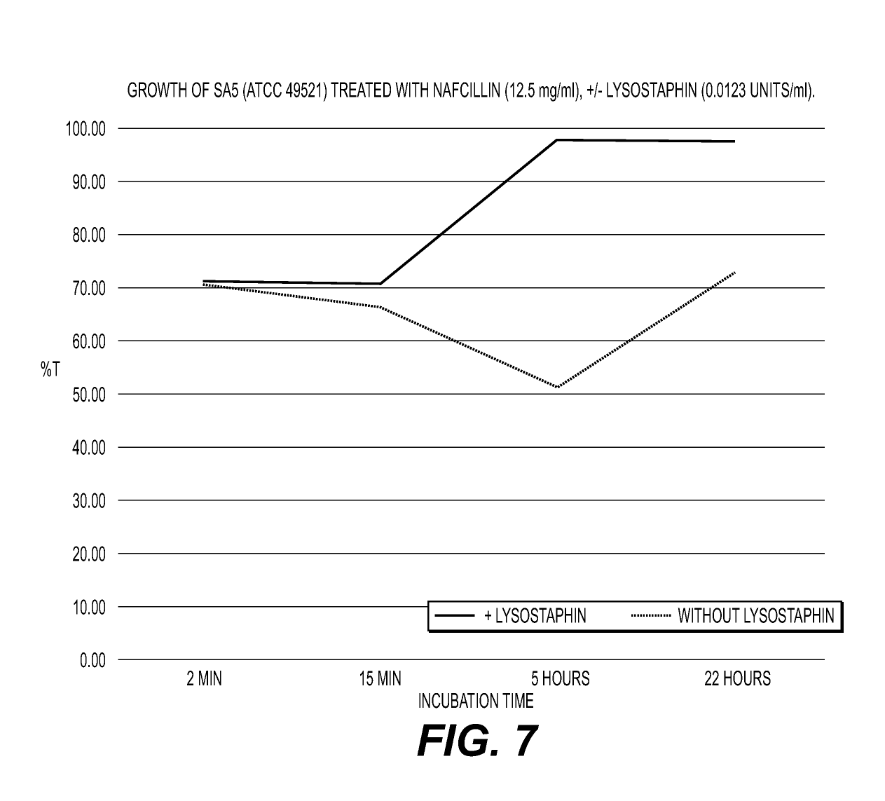

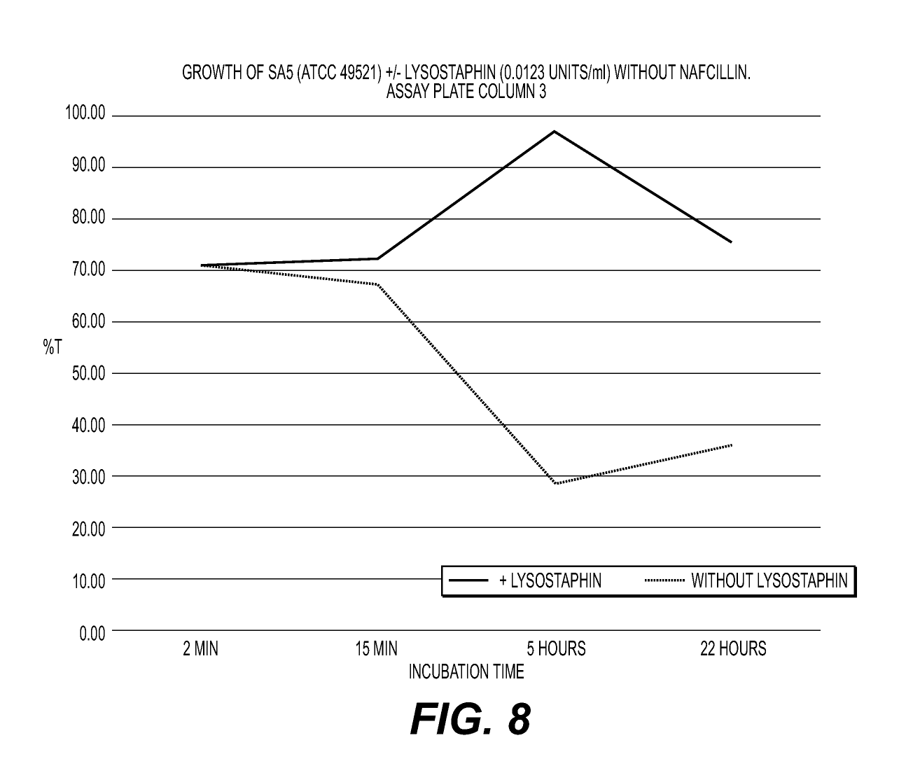

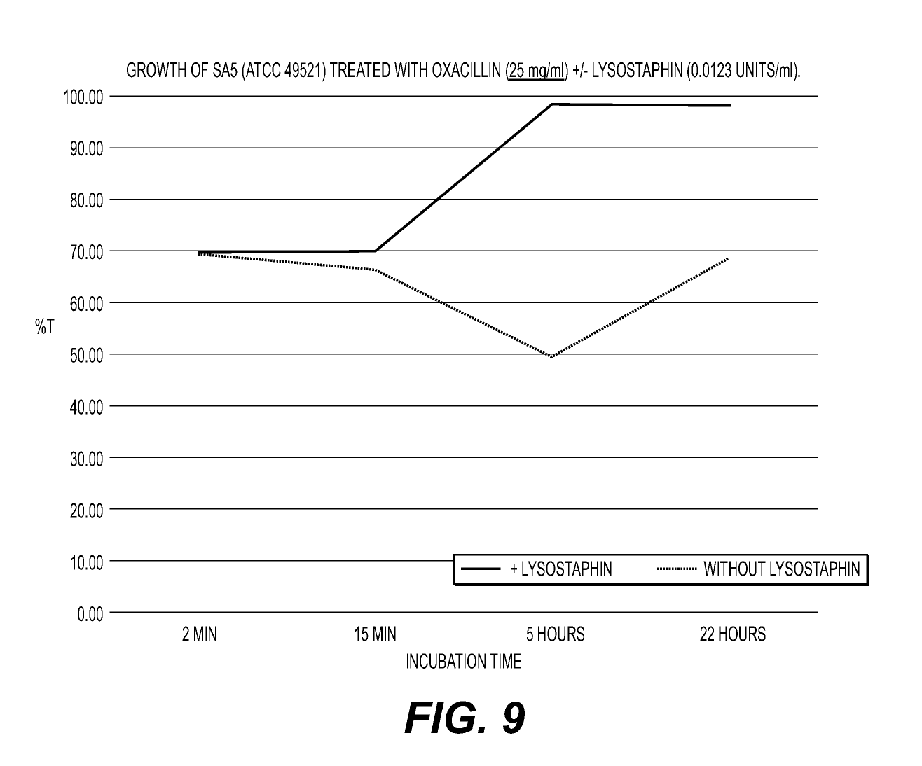

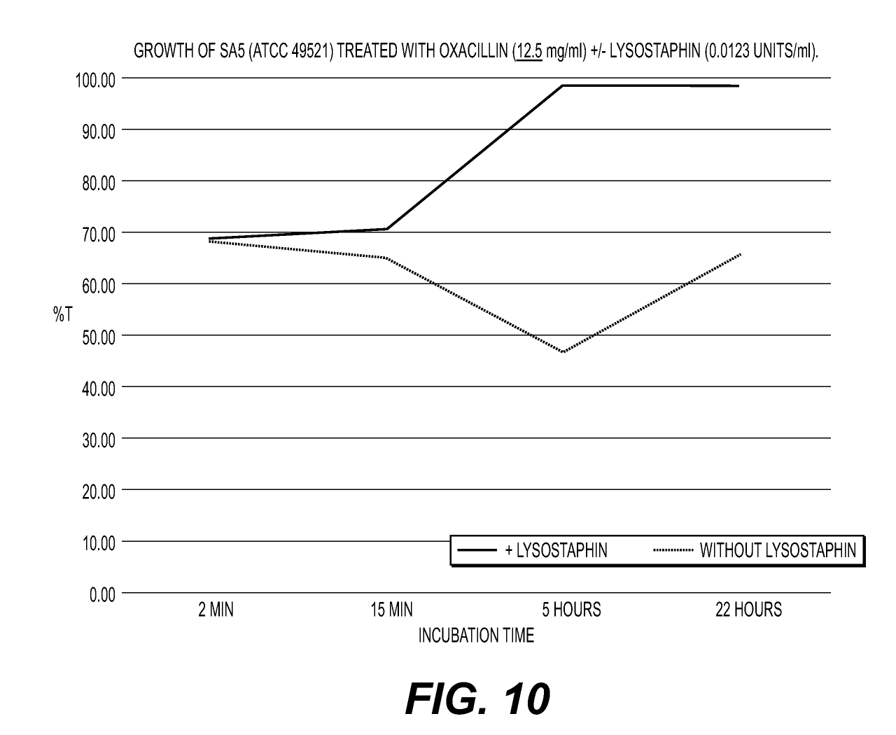

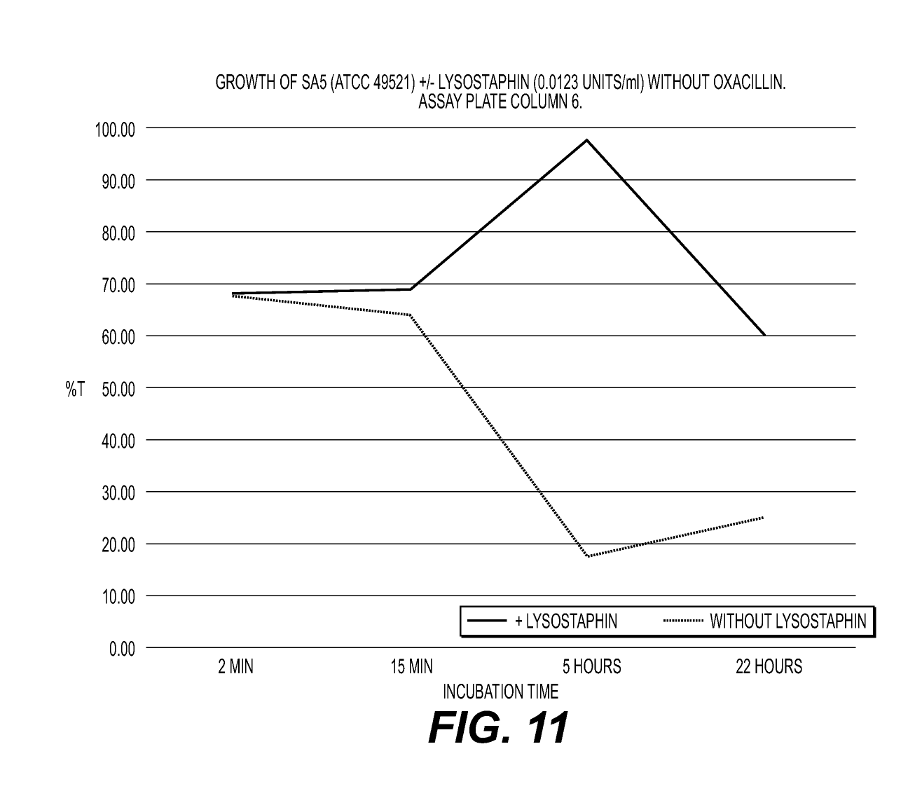

FIGS. 6-11 show the % T for each level of each antibiotic, +/−lysostaphin at 0.0123 units/ml. Lysostaphin at higher concentrations resulted in % T of >95% at 5 (not shown). After 22 hours the % T for most concentrations of lysostaphin (without antibiotics) was below 70%. Even at the 0.0123 units of lysostaphin per ml, the killing of the SA5 by lysostaphin alone was quite high within 5 hours even without antibiotics (FIGS. 8 and 11).

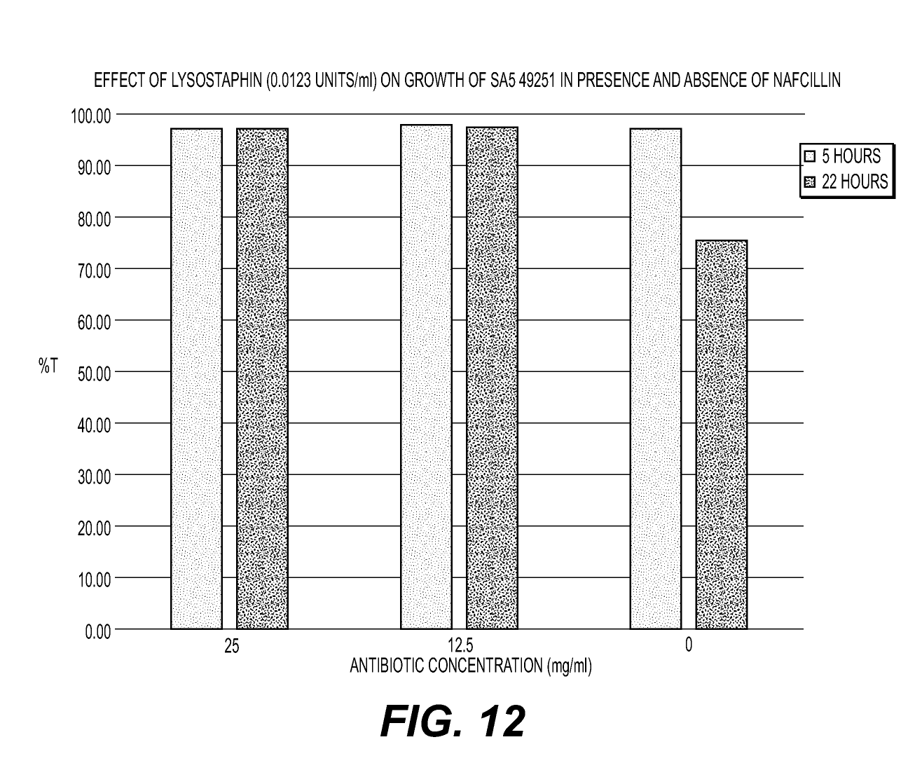

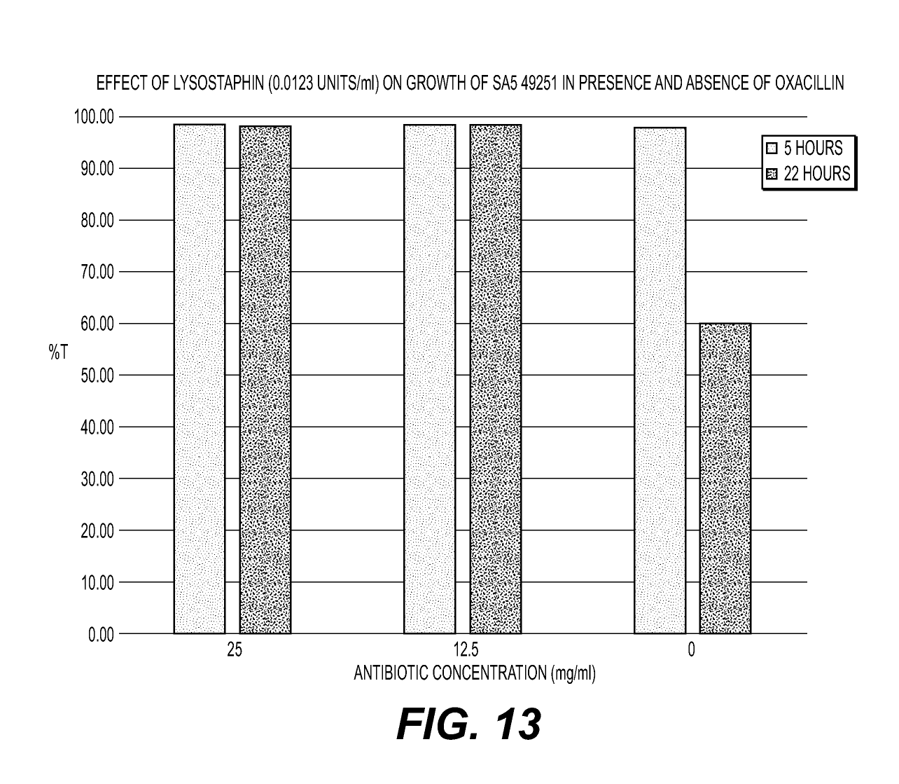

Between 5 hours and 22 hours, the % T with lysostaphin alone decreased, indicating that all of the bacteria were not killed and had started to grow during the incubation period. For example, in FIG. 8, the % T for the SA5 incubated with lysostaphin at 5 hours was 97.0 and that value fell to 75.4% T after 22 hours. In FIG. 11 the result is more dramatic, as the % T changed from 98.0 to 60.2. This decrease in % T was not evident when the antibiotics were included, indicating a synergistic effect between the antibiotics and lysostaphin (FIGS. 12 and 13). With either concentration of Nafcillin or Oxacillin, the % T remained over 98% even after 22 hours of incubation.

In this example the ultralow level of lysostaphin was reduced to just 0.0123 units/ml and most, but not all of the Staph were lysed and killed within 5 hours (>95% T). However, between 5 and 22 hours in the absence of antibiotics, the surviving bacteria again began to grow as evidenced by the lower % transmission. The staph grew at the high levels of oxacillin and nafcillin used in this study as demonstrated by the dropping % T at 5 hours and minimal increase by 22 hours, thus demonstrating ineffective rapid inhibition and killing of the staph. Surprisingly the ultralow level of lysostaphin acted synergistically with the antibiotics to provide rapid effective lysis and killing of the staph with the antibiotic preventing the resurgence of bacterial growth between 5 and 22 hours even when the antibiotics and lysostaphin were not totally effective alone.

Other embodiments and uses of the invention will be apparent to those skilled in the art from consideration of the specification and practice of the invention disclosed herein. All references cited herein, including all publications, U.S. and foreign patents and patent applications, and U.S. Application entitled “Multimodal Antimicrobial Therapy” filed contemporaneously herewith, are specifically and entirely incorporated by reference. The term comprising, where ever used, is intended to include the terms consisting and consisting essentially of. Furthermore, the terms comprising, including, containing and the like are not intended to be limiting. It is intended that the specification and examples be considered exemplary only with the true scope and spirit of the invention indicated by the following claims.

1. A pharmaceutical composition for administration to a patient in need thereof comprising a therapeutically effective amount of lysostaphin.

2. The composition of claim 1, wherein the effective amount of lysostaphin is administered at from 5 μg to 0.5 mg per kg of patient body weight.

3. The composition of claim 1, wherein the effective amount of lysostaphin provides a serum or tissue level of lysostaphin at from 0.001 μg/ml to 50 μg/ml.

4. The composition of claim 1, wherein the effective amount of lysostaphin provides a serum or tissue level of lysostaphin at from 0.01 μg/ml to 20 μg/ml.

5. The composition of claim 1, wherein the lysostaphin is wild-type lysostaphin isolated from Staphylococcus staphylolyticus.

6. The composition of claim 1, wherein the lysostaphin is a recombinant lysostaphin.

7. The composition of claim 6, wherein the recombinant lysostaphin is isolated from a culture of cells or microbes other than Staphylococcus cells.

8. The composition of claim 7, wherein the culture of cells other than Staphylococcus cells is a culture of E. coli cells.

9. The composition of claim 6, wherein the recombinant lysostaphin has one or more amino acids or one or more amino acid modifications that differ from wild-type lysostaphin.

10. The composition of claim 6, wherein the recombinant lysostaphin has a greater efficacy against Staphylococcus infection as compared to wild-type lysostaphin.

11. The composition of claim 6, wherein the recombinant lysostaphin has a reduced or no immune response when administered to the patients in need thereof as compared to wild-type lysostaphin.

12. The composition of claim 1, further comprising a secondary therapy for the patient in need thereof that is synergistic with the lysostaphin.

13. The composition of claim 12, wherein the secondary therapy comprises administration of antibodies or administration of antibiotics.

14. The composition of claim 13, wherein the antibodies are directed against Staphylococcus cells.

15. The composition of claim 13, wherein the antibiotics are one or more of chemical forms and derivatives of penicillin, amoxicillin, augmentin, polymyxin B, cycloserine, autolysin, bacitracin, cephalosporin, vancomycin, or beta lactam.

16. The composition of claim 13, wherein the antibiotics are administered to the patient in need thereof at a dose that is lower than the recommended dose for administration of the antibody alone.

17. The composition of claim 1, which is encapsulated or aerosolized.

18. The composition of claim 17, wherein the encapsulated composition is biodegradable and provides a slow-release or a timed-release of lysostaphin when administered to a patient.

19. The composition of claim 17, wherein the aerosolized composition comprises a particle size of about 1-3 microns or less.

20. The composition of claim 1, further comprising a pharmaceutically acceptable carrier.

21. The composition of claim 20, wherein the pharmaceutically acceptable carrier comprises one or more of oil, fatty acids, lipids, polymers, carbohydrates, gelatin, solvents, saccharides, buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents or an immunological inert substance, a carrier designated as generally recognized as safe (GRAS), or a combination thereof.

22. A method for treating or preventing a Staphylococcus infection comprising administering to a patient in need thereof a pharmaceutical composition comprising a therapeutically effective amount of lysostaphin.

23. The method of claim 22, wherein the lysostaphin is administered in one or more doses at from 1 μg to 1.0 mg per kg of patient body weight.

24. The method of claim 22, wherein the lysostaphin is administered in one or more doses and provides a serum or tissue level of lysostaphin from 0.001 μg/ml to 50 μg/ml.

25. The method of claim 22, wherein the lysostaphin is administered in one or more doses and provides a serum or tissue level of lysostaphin from 0.01 μg/ml to 20 μg/ml.

26. The method of claim 22, wherein the composition is administered orally, intravenously or subcutaneously.

27. The method of claim 22, wherein the composition is encapsulated with a biodegradable polymer that provides for slow-release or timed-release of lysostaphin.

28. The method of claim 22, wherein the lysostaphin is aerosolized to an average particle size of about 1-3 microns or less.

29. The method of claim 22, wherein the composition is coated onto an object to be inserted into the body of the patient.

30. The method of claim 29, wherein after administration of the object, a serum or tissue level of lysostaphin is from 0.001 μg/ml to 50 μg/ml.

31. The method of claim 30, wherein the serum or tissue level of lysostaphin is from 0.01 μg/ml to 20 μg/ml.

32. The method of claim 29, wherein the object is inserted into an area of the body of the patient that is sequestered from the patient's immune system.

33. The method of claim 22, wherein the composition has one or more reduced negative effects or one or more increased positive effects for the patient as compared with conventional therapy.

34. The method of claim 33, wherein the reduced negative effects include one or more of reduced toxicity and reduced immunogenicity.

35. The method of claim 33, wherein the enhanced positive effects include one or more of increased efficacy and enhanced clearance from a patient system.

36. The method of claim 22, wherein the lysostaphin is wild-type or recombinant lysostaphin.
